SEQUENCE LISTING (1 sequence)

SEQ ID NO: 1 - DNA, 20 bp, Artificial Sequence
  Synthetic oligonucleotide
tgtcatattcctggatcctt

(19) United States
(12) Patent Application Publication
(10) Pub. No.: US 2014/0323543 A1
(43) Pub. Date: Oct. 30, 2014

(54) Treatment of Prostate Cancer with eIF4E Antisense Compounds

(71) Applicant: Jeremy Richard Graff, Indianapolis, IN (US)

(72) Inventor: Jeremy Richard Graff, Indianapolis, IN (US)

(21) Appl. No.: 13/870,823

(22) Filed: Apr. 25, 2013

(51) Int. Cl. C12N 15/113

(52) U.S. Cl. CPC C12N 15/113
USPC 514/44 A

(57) ABSTRACT

The present invention provides methods of sensitizing cancer cells to docetaxel and inhibiting the growth of various tumors by employing a modified eIF-4E antisense oligonucleotide and docetaxel in combination.

The present invention relates to the use of an antisense oligonucleotide (ASO) therapeutic targeted to eukaryotic translation initiation factor 4E (eIF-4E) in combination with docetaxel (Taxotere®) in order to achieve an enhanced therapeutic effect in treating cancers.

eIF-4E is elevated in multiple human cancers and is directly related to disease progression. Elevated eIF-4E function triggers enhanced assembly of the eIF-4F translation initiation complex, which includes eIF-4E as a component, driving the expression of a pool of mRNAs that are exceptionally dependent on elevated eIF-4F activity for translation and which promote tumor cell growth, proliferation, development, survival, and angiogenesis. In view of the involvement of eIF-4E in tumor growth and development, this protein is an attractive therapeutic target.

PCT International Publication WO 2005/028628 discloses ASOs targeted to eIF-4E, including the ASO disclosed herein, and methods of using these ASOs for modulating the expression or overexpression of eIF-4E in vitro and In vivo.

Graff et al. (2007) J. Clin. Invest. 117:2638-48 discloses studies on a number of ASOs targeted to eIF-4E, also including the ASO disclosed herein.

Advanced, hormone refractory prostate cancer is most often treated with Taxotere® (Bradley and Hussain (2008) Cancer J. 14(1): 15-19). Other cancers, including breast (Metro et al. (2008) Anticancer Res. 28(2B): 1245-58) and non-small cell lung cancers (Stinchcombe et al. (2008) Oncologist 13 (Suppl. 1):28-36) are also commonly treated with Taxotere®.

There exists a need for improved therapies for the treatment of cancers. The combined use of the chemically modified eIF-4E ASO disclosed herein, which has been shown to selectively reduce gene and protein expression of eIF-4E, with docetaxel in 3 different human xenografted cancer models variously results in an additive or greater-than-additive effect in reducing tumor volume compared to the sum of the tumor volume-reducing activity achieved with either agent alone. The chemical modifications of the ASO enhance resistance to degradation by cellular nucleases and increase nucleic acid duplex stability with the eIF-4E mRNA target. The high target selectivity of the ASO minimizes non-target related undesired and adverse side effects. Both docetaxel and the ASO can be administered systemically and distribute throughout the body. The combination of the ASO and docetaxel is therefore a potent and effective therapeutic combination of high selectivity in cancer cells. In addition, the drug combinations disclosed herein possess other highly desirable pharmacologic properties, such as high bioavailability associated with intravenous administration, good in vivo metabolic stability, and pharmacokinetic/pharmacodynamic properties that permit convenient dosing.

Thus, among its various aspects, the present invention provides:

A method of treating lung cancer, prostate cancer, or breast cancer, comprising administering to a patient in need thereof a therapeutically effective combination of docetaxel and a modified eIF-4E antisense oligonucleotide which is in the form of a pharmaceutically acceptable salt. The pharmaceutically acceptable salt can be a sodium salt. In an embodiment of this aspect of the invention, docetaxel is administered after administration of the modified eIF-4E antisense oligonucleotide, within a therapeutically effective interval. In a further embodiment, the therapeutically effective interval is in the range of from about 4 days to about 21 days.

A method of treating lung cancer, prostate cancer, or breast cancer, comprising administering to a patient in need thereof a modified eIF-4E antisense oligonucleotide which is in the form of a pharmaceutically acceptable salt, and docetaxel in amounts that in combination are effective in treating said lung cancer, prostate cancer, or breast cancer. The pharmaceutically acceptable salt can be a sodium salt. In an embodiment of this aspect of the invention, docetaxel is administered after administration of the modified eIF-4E antisense oligonucleotide, within a therapeutically effective interval. In a further embodiment, the therapeutically effective interval is in the range of from about 4 days to about 21 days.

A method of treating lung cancer, prostate cancer, or breast cancer, comprising sequentially administering to a patient in need thereof an amount of a modified eIF-4E antisense oligonucleotide consisting of the nucleotide sequence which is in the form of a pharmaceutically acceptable salt, followed by an amount of docetaxel,
wherein said amounts of said modified eIF-4E antisense oligonucleotide and said docetaxel in combination are effective in treating said lung cancer, prostate cancer, or breast cancer in said patient, and
wherein said docetaxel is administered within a therapeutically effective interval after administration of said modified eIF-4E antisense oligonucleotide. The pharmaceutically acceptable salt can be a sodium salt. In an embodiment of this aspect of the invention, the therapeutically effective interval is in the range of from about 4 days to about 21 days.

A method of treating non-small cell lung cancer, comprising:
administering to a patient in need thereof a docetaxel-sensitizing amount of a modified eIF-4E antisense oligonucleotide which is in the form of a pharmaceutically acceptable salt, followed by
administering to said patient an effective amount of docetaxel within a therapeutically effective interval,
wherein together said amounts result in an additive percent reduction in the volume of a non-small cell lung cancer tumor compared to the sum of the percent reductions in the volume of said non-small cell lung cancer tumor achieved by administering said modified eIF-4E antisense oligonucleotide alone and said docetaxel alone. The pharmaceutically acceptable salt can be a sodium salt. In an embodiment of this aspect of the invention, the therapeutically effective interval is in the range of from about 4 days to about 21 days.

A method of treating prostate cancer, comprising:
administering to a patient in need thereof a docetaxel-sensitizing amount of a modified eIF-4E antisense oligonucleotide which is in the form of a pharmaceutically acceptable salt, followed by
administering to said patient an effective amount of docetaxel within a therapeutically effective interval,
wherein together said amounts result in an additive percent reduction in the volume of a prostate cancer tumor compared to the sum of the percent reductions in the volume of said prostate cancer tumor achieved by administering said modified eIF-4E antisense oligonucleotide alone and said docetaxel alone. The pharmaceutically acceptable salt can be a sodium salt. In an embodiment of this aspect of the invention, the therapeutically effective interval is in the range of from about 4 days to about 21 days.

A method of treating breast cancer, comprising:
administering to a patient in need thereof a docetaxel-sensitizing amount of a modified eIF-4E antisense oligonucleotide which is in the form of a pharmaceutically acceptable salt, followed by
administering to said patient an effective amount of docetaxel within a therapeutically effective interval,
wherein together said amounts result in a greater-than-additive percent reduction in the volume of a breast cancer tumor compared to the sum of the percent reductions in the volume of said breast cancer tumor achieved by administering said modified eIF-4E antisense oligonucleotide alone and said docetaxel alone. The pharmaceutically acceptable salt can be a sodium salt. In an embodiment of this aspect of the invention, the therapeutically effective interval is in the range of from about 4 days to about 21 days.

A method of sensitizing a non-small cell lung cancer cell, a prostate cancer cell, or a breast cancer cell to docetaxel, comprising:
contacting said non-small cell lung cancer cell, prostate cancer cell, or breast cancer cell and a docetaxel-sensitizing amount of a modified eIF-4E antisense oligonucleotide consisting of the nucleotide sequence which is in the form of a pharmaceutically acceptable salt, and
subsequently contacting said non-small cell lung cancer cell, prostate cancer cell, or breast cancer cell and an effective amount of docetaxel within an effective interval. The pharmaceutically acceptable salt can be a sodium salt. In an embodiment of this aspect of the invention, the effective interval is in the range of from about 4 days to about 21 days.

A method of inhibiting the growth or proliferation of a non-small cell lung cancer cell, a prostate cancer cell, or a breast cancer cell, comprising contacting said non-small cell lung cancer cell, prostate cancer cell, or breast cancer cell with:
an amount of a modified eIF-4E antisense oligonucleotide which is in the form of a pharmaceutically acceptable salt, and
an amount of docetaxel,
wherein said amounts of said modified eIF-4E antisense oligonucleotide and said docetaxel are effective in combination in inhibiting the growth or proliferation of said non-small cell lung cancer cell, prostate cancer cell, or breast cancer cell. The pharmaceutically acceptable salt can be a sodium salt. In an embodiment of this aspect of the invention, the non-small cell lung cancer cell, prostate cancer cell, or breast cancer cell is first contacted with the modified eIF-4E antisense oligonucleotide, and subsequently with docetaxel, within an effective interval. In a further embodiment, the effective interval is in the range of from about 4 days to about 21 days.

A method of inhibiting the growth or proliferation of a non-small cell lung cancer cell or a prostate cancer cell, comprising:
contacting said non-small cell lung cancer cell or prostate cancer cell and a docetaxel-sensitizing amount of a modified eIF-4E antisense oligonucleotide r, nucleotides 6-15 are 2′-deoxynucleotides, every cytosine residue is a 5-methyl-cytosine, and which is in the form of a pharmaceutically acceptable salt, followed by
contacting said non-small cell lung cancer cell or prostate cancer cell and an effective amount of docetaxel within an effective interval,
wherein together said amounts produce an additive effect in inhibiting the growth or proliferation of said non-small cell lung cancer cell or prostate cancer cell compared to the sum of the effect achieved with said modified eIF-4E antisense oligonucleotide alone and said docetaxel alone. The pharmaceutically acceptable salt can be a sodium salt. In an embodiment of this aspect of the invention, the effective interval is in the range of from about 4 days to about 21 days.

A method of inhibiting the growth or proliferation of a breast cancer cell, comprising:
contacting said breast cancer cell and a docetaxel-sensitizing amount of a modified eIF-4E antisense oligonucleotide which is in the form of a pharmaceutically acceptable salt, followed by
contacting said breast cancer cell and an effective amount of docetaxel within an effective interval,
wherein together said amounts produce a greater-than-additive effect in inhibiting the growth or proliferation of said breast cancer cell compared to the sum of the effect achieved with said modified eIF-4E antisense oligonucleotide alone and said docetaxel alone. The pharmaceutically acceptable salt can be a sodium salt. In an embodiment of this aspect of the invention, the effective interval is in the range of from about 4 days to about 21 days.

A method of inhibiting lung, prostate, or breast tumor growth, comprising administering to a patient in need thereof a modified eIF-4E antisense oligonucleotide which is in the form of a pharmaceutically acceptable salt, and docetaxel in amounts that in combination are effective in inhibiting growth of said lung, prostate, or breast tumor. The pharmaceutically acceptable sail can be a sodium salt. In an embodiment of this aspect of the invention, docetaxel is administered after administration of the modified eIF-4E antisense oligonucleotide, within a therapeutically effective interval. In a further embodiment, the therapeutically effective interval is in the range of from about 4 days to about 21 days.

A method of inhibiting increase in tumor volume of a non-small cell lung cancer tumor or a prostate cancer tumor, comprising:
administering to a patient in need thereof a docetaxel-sensitizing amount of a modified eIF-4E antisease oligonucleotide which is in the form of a pharmaceutically acceptable salt, followed by
administering an effective amount of docetaxel within a therapeutically effective interval,
wherein together said amounts produce an additive percent reduction in inhibiting tumor volume increase of said non-small cell lung cancer tumor or prostate cancer tumor compared to the sum of the percent reductions in tumor volume increase achieved by administering said modified eIF-4E antisense oligonucleotide alone and said docetaxel alone. The pharmaceutically acceptable salt can be a sodium salt. In an embodiment of this aspect of the invention, the therapeutically effective interval is in the range of from about 4 days to about 21 days.

A method of inhibiting increase in tumor volume of a breast cancer tumor, comprising:
administering to a patient in need thereof a docetaxel-sensitizing amount of a modified eIF-4E antisense oligonucleotide which is in the form of a pharmaceutically acceptable salt, followed by
administering an effective amount of docetaxel within a therapeutically effective interval,
wherein together said amounts produce a greater-than-additive percent reduction in inhibiting tumor volume increase of said breast cancer tumor compared to the sum of the percent reductions in tumor volume increase achieved by administering said modified eIF-4E antisense oligonucleotide alone and said docetaxel alone. The pharmaceutically acceptable salt can be a sodium salt. In an embodiment of this aspect of the invention, the therapeutically effective interval is in the range of from about 4 days to about 21 days.

A method of enhancing the therapeutic effectiveness of docetaxel in treating lung cancer, prostate cancer, or breast cancer, comprising administering to a patient in need thereof a therapeutically effective combination of (1) a modified eIF-4E antisense oligonucleotide consisting of the nucleotide sequence which is in the form of a pharmaceutically acceptble salt, and (2) docetaxel. The pharmaceutically acceptable salt can be a sodium salt. In an embodiment of this aspect of the invention, docetaxel is administered after administration of the modified eIF-4E antisense oligonucleotide, within a therapeutically effective interval. In a further embodiment, the therapeutically effective interval is in the range of from about 4 days to about 21 days.

Use of the compound of formula I:

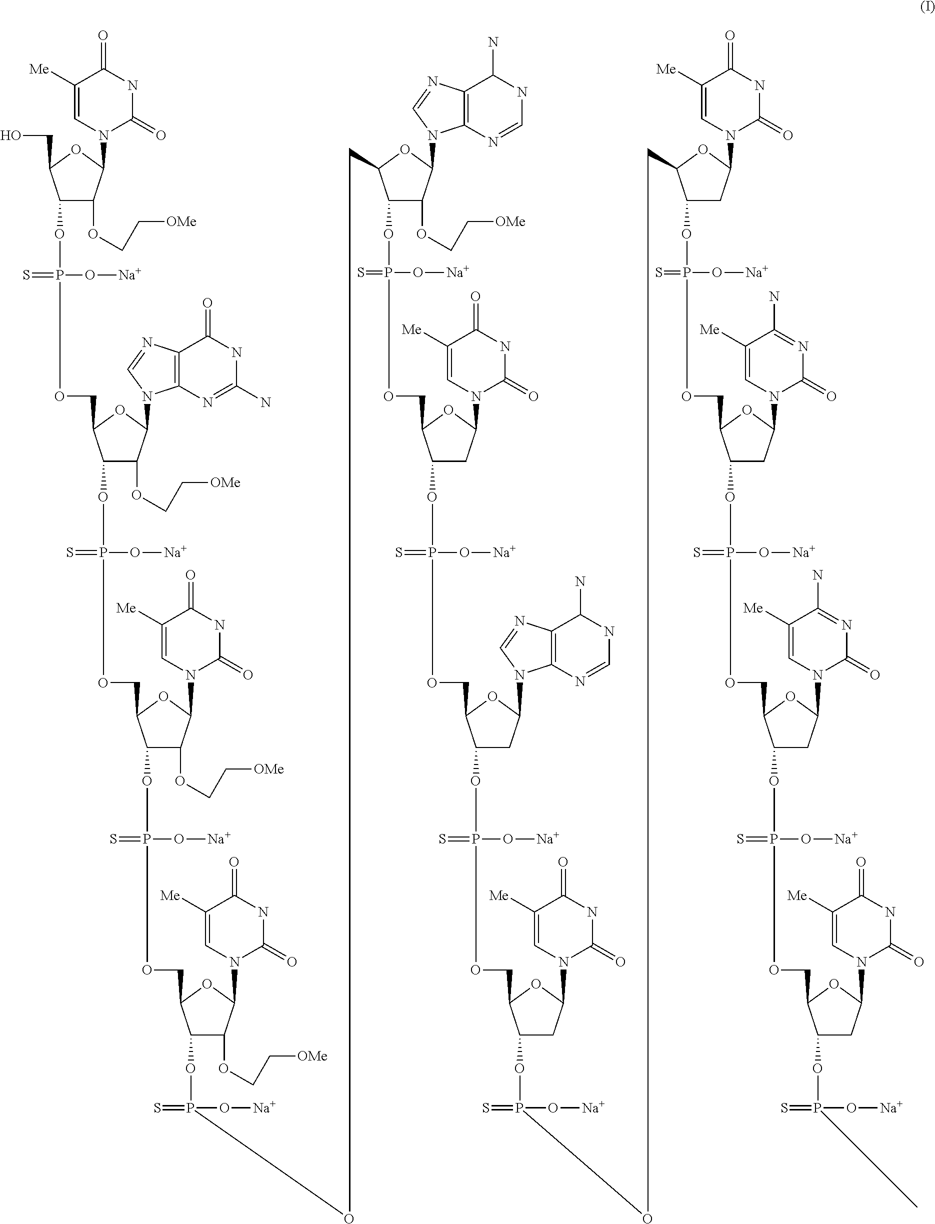

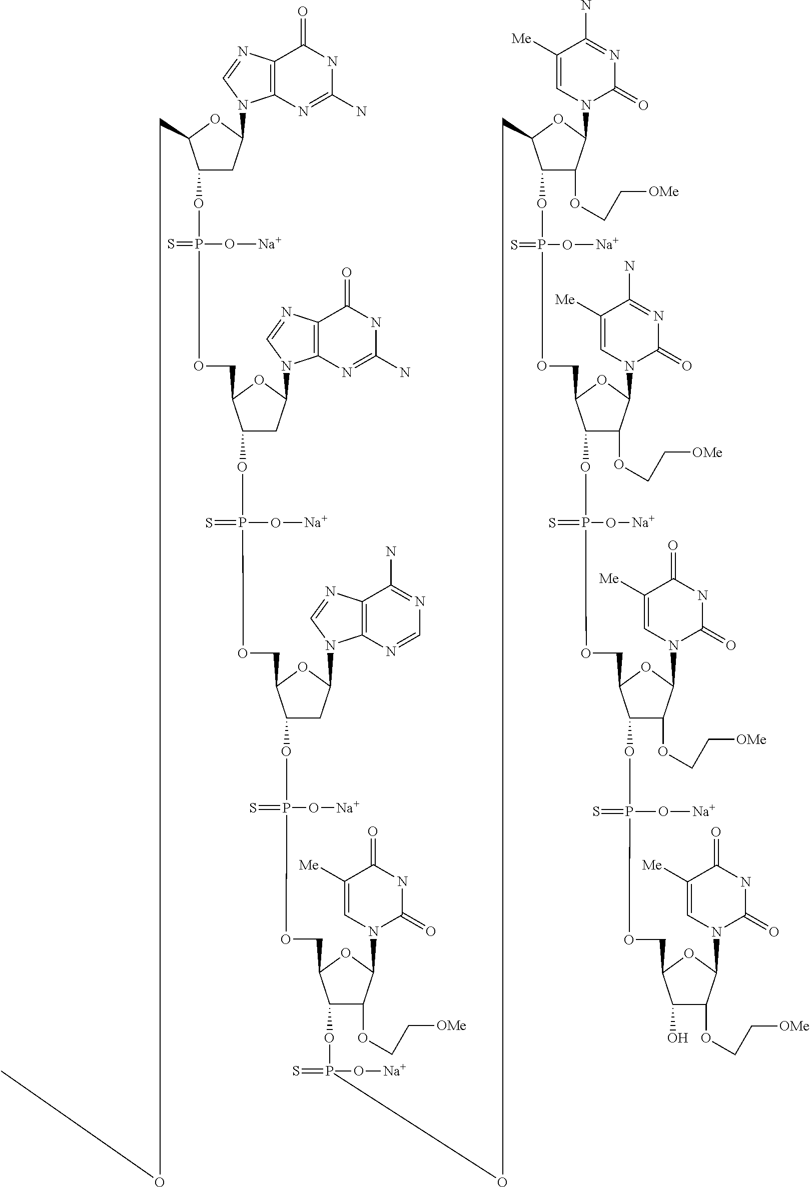

or other pharmaceutically acceptable salt thereof in the manufacture of a medicament for breast cancer in a patient, wherein said medicament is to be administered in combination with docetaxel. In an embodiment of this aspect of the invention, the compound of formula I, or other pharmaceutically acceptable salt thereof, and docetaxel are administered separately, within a therapeutically effective interval. In a further embodiment, the docetaxel is administered after the compound of formula I, or other pharmaceutically acceptable salt thereof, within a therapeutically effective interval. In a further embodiment, the therapeutically effective interval is in the range of from about 4 days to about 21 days. In further embodiments of any of these uses, the combination therapy is via the parenteral route, preferably via intravenous administration, more preferably via slow infusion. In a further embodiment of any of these uses, each of the compound of formula I, or other pharmaceutically acceptable salt thereof, and docetaxel is in the form of a sterile injectable solution.

A product containing the compound of formula I:

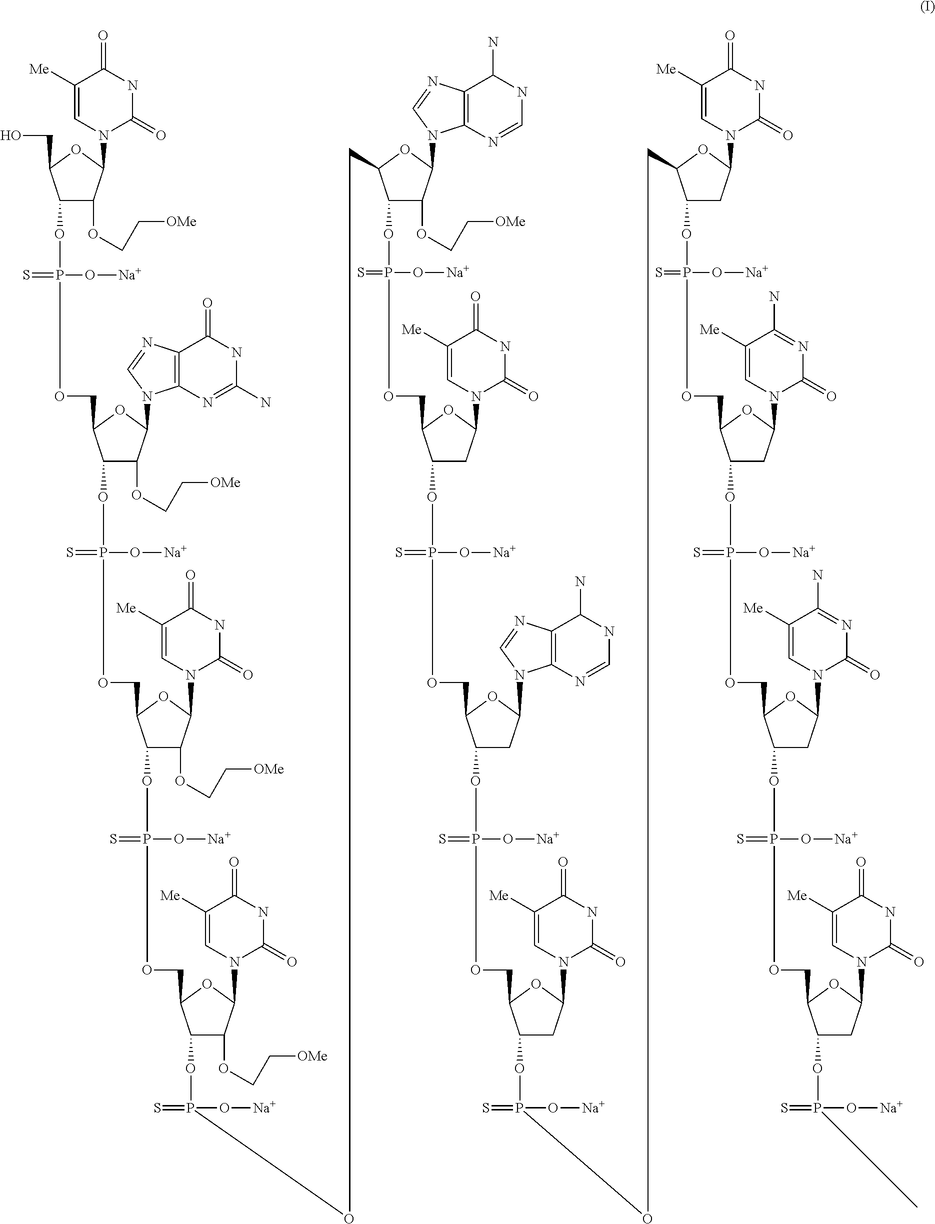

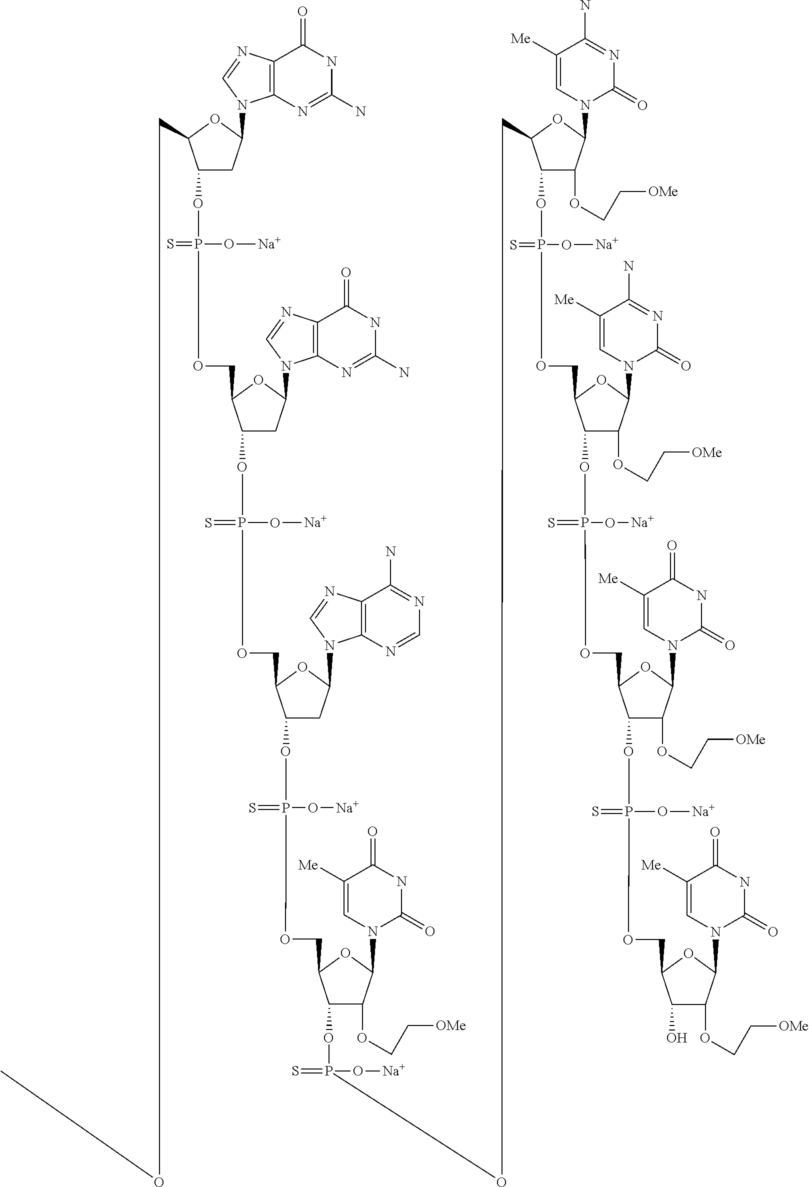

or other pharmaceutically acceptable salt thereof and docetaxel as a combined preparation for simultaneous or separate use in treating non-small cell lung cancer, prostate cancer, or breast cancer. In an embodiment of this aspect of the invention, each of the compound of formula I, or other pharmaceutically acceptable salt thereof, and docetaxel is in the form of a sterile injectable solution.

Tables 1-3 show the effect of combined administration of the eIF-4E ASO and docetaxel on mean tumor volume of human non-small cell lung cancer, hormone-refractory prostate cancer, and breast cancer xenografts in mice. The data indicate that the combination treatment variously results in an additive or greater-than-additive percent reduction in mean tumor volume depending upon the tumor type (additive: lung and prostate; greater-than-additive: breast) compared to the sum of the percent reduction achieved via treatment with either drug agent alone.

### Combination Therapy in Cancer

Clinical protocols in cancer chemotherapy commonly employ multiple drugs rather than a single therapeutic. When each drug alone exhibits inhibitory effects, the combined effect may be antagonism, additivity, or synergism. If one of the drugs has no effect by itself but increases the effects of other drug(s), the result is called potentiation. Prediction of synergy is difficult. Each drug in combination has its own effects, i.e., its own potency and a specific shape of dose-effect curve. These effects are also related to affinity and efficacy. Factors such as feedback inhibition, spatial, temporal, and microenvironmental factors (such as pH, ionic strength, and temperature) add to the biological complexity and intricacy of drug effects. Further factors complicating prediction of synergy include individual drug absorption, permeability, transport, and metabolic activation and inactivation. Hypotheses of the possible occurrence of synergism ultimately require confirmation by experimental findings (Rideout and Chou (1991) in Chou and Rideout, Eds., Synergism and Antagonism in Chemotherapy, Academic Press, Inc., New York, pp. 6 and 21). Prediction of additivity is similarly difficult, and antagonism is always a possibility when multiple drugs are administered in combination.

Mizushima et al. ((1995) Anticancer Res. 15(1):37-43) disclose that investigators should use caution when using antisense oligonucleotides for chemosensitization of cells. Pretreatment with antisense oligonucleotides can at times have a tendency to reduce rather than enhance drug cytotoxicity. This may be caused by a number of nonspecific oligonucleotide effects, such as the ability of the oligonucleotide to interact directly with the cytotoxic drug (Blagosklonny et al. (1994) Anticancer Drugs 5(4):437-442).

In view of all the foregoing factors, demonstration by the present inventor of the docetaxel-sensitizing effect of the eIF-4E ASO in cancer cells and the additive or greater-than-additive effect in inhibiting cancer cell proliferation and tumor growth achieved by the combined use of the eIF-4E ASO and docetaxel are novel, surprisingly unexpected, and therapeutically useful.

### DEFINITIONS

The term “eIF-4E antisense oligonucleotide” or “eIF-4E ASO” as used herein refers to the eIF-4E antisense oligonucleotide originally described in WO 2005/028628 and known by the chemical name: d(P-thio)([2′-O-(2-methoxyethyl)]m5rU-([2′-O-(2-methoxyethyl)]rG-([2′-O-(2-methoxyethyl)]m5rU-([2′-O-(2-methoxyethyl)]m5rC-([2′-O-(2-methoxyethyl)]rA-T-A-T-T-m5C-m5C-T-G-G-A-([2′-O-(2-methoxyethyl)]m5rU-([2′-O-(2-methoxyethyl)]m5rC-([2′-O-(2-methoxyethyl)]m5rC-([2′-O-(2-methoxyethyl)]m5rU-([2′-O-(2-methoxyethyl)]m5rU) which is in the form of a pharmaceutically acceptable salt, preferably an alkali metal salt, more preferably a lithium, sodium, or potassium salt, and most preferably a sodium salt. The chemical structure of the latter is:

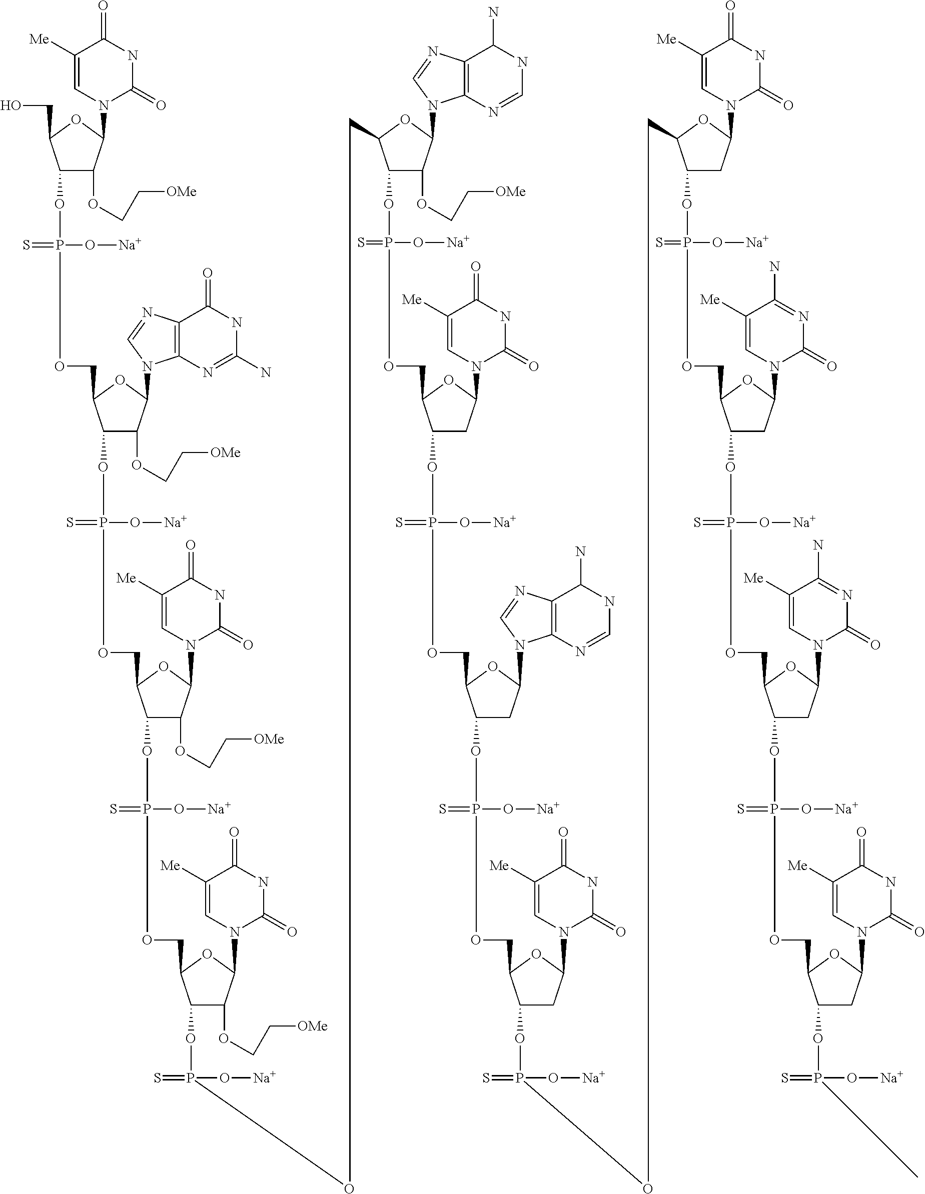

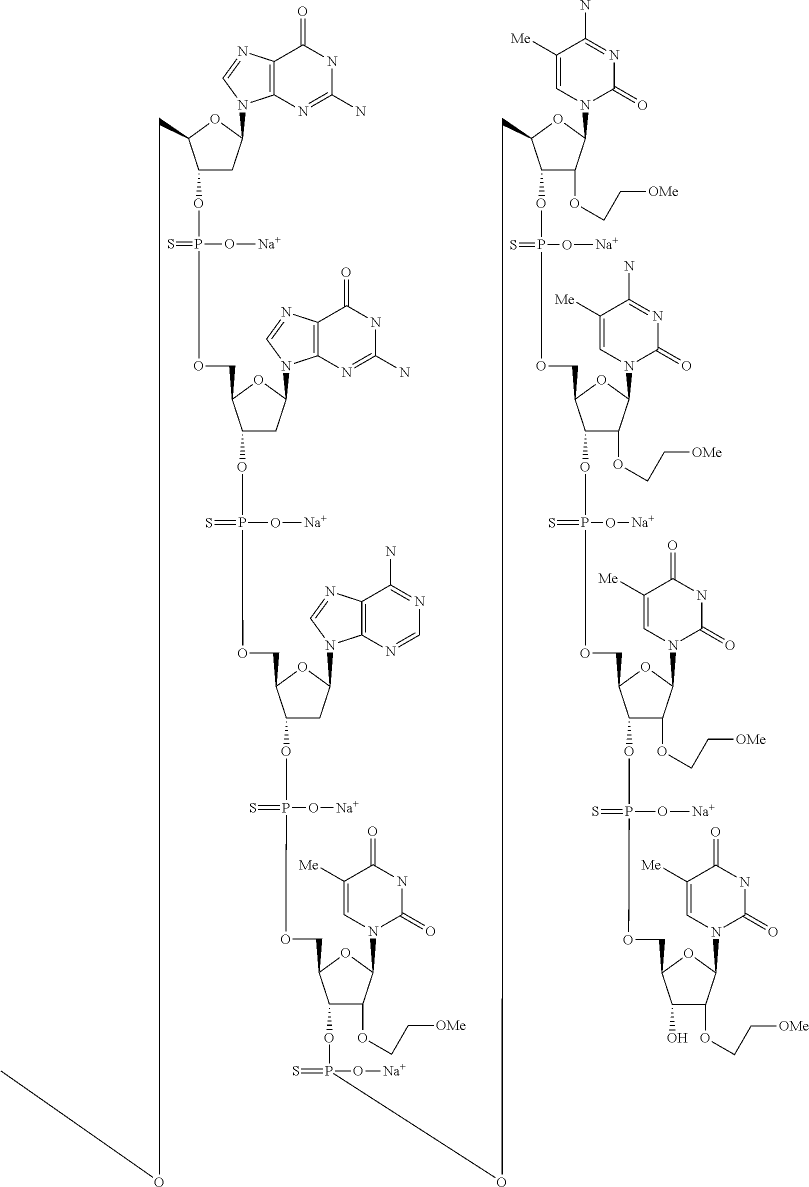

The eIF-4E ASO is described in PCT International Publication WO 2005/02828.

Doctaxel is an antineoplastic agent belonging to the taxoid family. Its chemical name is (2R,3S)—N-carboxy-3-phenylisoseine,N-tert-butyl ester, 13-ester with 5β-20-epoxy-1,2α,4,7β,10β,13α-hexahydroxytax-11-en-9-one 4-acetate 2-benzoate, trihydrate.

The structure of docetaxel is:

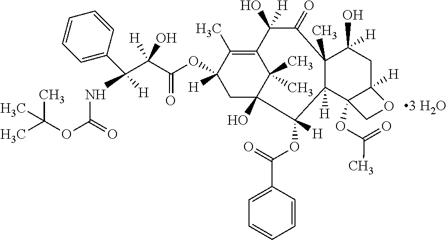

Docetaxel is described in U.S. Pat. No. 4,814,470, which discloses the synthesis, formulation, and methods of using this compound for the treatment of susceptible neoplasms. The Physicians' Desk Reference ((2008), Edition 62, Thomson Healthcare Inc., Montvale, N.J., pp. 2883-2895) provides further guidance concerning the therapeutic use of docetaxel, including the concomitant use of a corticosteroid, for example dexamethasone or prednisone. As disclosed therein, docetaxel disrupts the microtubular network in cells, inhibiting mitosis and cell proliferation.

As used herein, the term “patient” refers to a mammal afflicted with one or more disorders associated with eIF-4E expression or overexpression. The most preferred patient is a human.

The term “treating” (or “treat” or “treatment”) refers to curative treatment of disorders associated with eIF-4E activity, including various cancers. Curative treatment refers to processes involving a slowing, interrupting, arresting, controlling, stopping, reducing, or reversing the progression or severity of a symptom, disorder, condition, or disease, but does not necessarily involve a total elimination of all disease-related symptoms, disorders, or conditions, or the disease itself.

“Inhibiting” means restraining, retarding, restricting, reducing, holding back, or preventing.

The phrase “sensitizing to docetaxel” and the like in connection with the eIF-4E ASO means making responsive to, susceptible to the action(s) of, or readily or easily affected by, docetaxel. In some cases, this can also mean eliciting a greater response to a dose or amount of docetaxel than that which would occur in the absence of the ASO.

The term “docetaxel-sensitizing amount” refers to an amount or dose of the eIF-4E ASO that is effective in making cancer cells responsive to, susceptible to the action(s) of, or readily or easily affected by, docetaxel, or eliciting a greater cancer cell response to the action of an amount or dose of docetaxel than that which would occur in response to this amount or dose of docetaxel in the absence of the eIF-4E ASO.

Therapeutically effective amounts of the eIF-4E ASO, which include docetaxel-sensitizing amounts in the therapeutic context, are in the range of from about 100 mg to about 1,200 mg in humans. A preferred dose in terms of efficacy and tolerability is about 1,000 to about 1,200 mg per single dose or administration, administered parenterally, preferably intravenously, more preferably via slow intravenous infusion, over 1-3 hours.

“Effective amount of docetaxel” refers to an amount or dose of docetaxel, when used in combination with the eIF-4E ASO, that produces the particular cancer cell growth- or proliferation inhibiting effect, tumor growth inhibiting effect, tumor volume increase-inhibiting effect, or cancer treatment effect in a cancer cell or tumor.

Therapeutically effective amounts of docetaxel are in the range of from about 60 to about 100 mg/m², preferably from about 75 to about 100 mg/m², per single dose or administration, administered parenterally, preferably intravenously, more preferably via slow intravenous infusion, over 1 hour weekly or every 3 weeks. A dose of 75 mg/m² is preferred.

In general, optimum dosages of each of these therapeutic agents can vary depending on the relative potency of the active ingredients in individual patients. Medical practitioners can determine dose and repetition rates for dosing based on measured residence times and concentrations of the active ingredients in bodily fluids or tissues and/or monitoring of relevant disease-related biomarkers for particular cancers.

In view of the additive and greater-than-additive treatment effects disclosed herein achieved via the combined use of the eIF-4E ASO and docetaxel, it is expected that subclinically effective amounts of docetaxel, compared to those when this drug is used alone, will be therapeutically effective in the methods of combination therapy disclosed herein. For any individual patient, therapeutically effective amounts of the eIF-4E ASO and docetaxel when used in combination can be determined by the healthcare provider by monitoring the effect of the combination on a relevant cancer biomarker. In the case of lung cancer, relevant biomarkers can be assessed by chest radiography, computed tomography (CT), low-dose spiral CT evaluation, magnetic resonance imaging (MRI), gallium scanning (scintigraphy), or position emission tomographic (PET) scanning. For prostate cancer. Prostate Specific Antigen (PSA) can be monitored. For breast cancer, monitoring can be performed by mammography, digital mammography, ultrasonography, thermography, light scanning, MRI, or by measuring carcinoembryonic antigen (CEA) or MUC1 levels in blood. Analysis of the data obtained by these methods permits modification of the treatment regimen during therapy so that optimal amounts of the eIF-4E ASO and docetaxel in combination therapy are administered, and so that the duration of treatment can be determined as well. In this way, the dosing/treatment regimen can be modified over the course of therapy so that the lowest amounts of the eIF-4E ASO and docetaxel used in combination that exhibit satisfactory therapeutic effectiveness are administered, and so that administration of these compounds is continued only so long as is necessary to successfully treat the patient.

The term “effective interval” is a period of time beginning upon contact of a cancer cell and the eIF-4E ASO and during which the cell is responsive to the anti-mitotic and cell growth- and proliferation-inhibiting effects of the combination of the ASO and docetaxel. This effect can be manifested by: (a) sensitization of the cancer cell to the effects of docetaxel: (b) inhibition of growth or proliferation of the cancer cell; (c) inhibition of tumor growth; (d) inhibition of increase in tumor volume; or (d) therapeutically enhanced cancer treatment effect, in a cancer cell or tumor. In the case of the non-small cell lung cancer, prostate cancer, and breast cancer tumor xenografts disclosed herein, the effective interval is initially 8 days.

The therapeutically effective eIF-4E ASO/docetaxel combination therapies disclosed herein can be achieved by separate administration of the eIF-4E ASO and docetaxel. The ASO can be administered first, followed by administration of docetaxel within a therapeutically effective interval. When administered separately, the eIF-4E ASO and docetaxel can be introduced into the patient on different schedules, as long as the time between the two administrations falls within a therapeutically effective interval. A “therapeutically effective interval” is a period of time after administration of the eIF-4E ASO to a patient during which a tumor is responsive to the beneficial anti-neoplastic therapeutic effects of the combination of the ASO and docetaxel. For example, the initial therapeutically effective interval upon commencement of patient therapy can include a pre-docetaxel treatment period comprising administration of 3 loading doses of the eIF-4E ASO, i.e., a 1-3 hour infusion of the eIF-4E ASO once per day for 3 consecutive days during the first week, followed by administration of at least I weekly maintenance dose of the ASO for an additional 2 weeks. After 21 days of administration of the eIF-4E ASO in this manner, docetaxel can then be administered. Thus, the initial therapeutically effective interval can be 21 days from initiation of treatment with the eIF-4E ASO, which includes 3 loading doses during the first week followed by 2 once-weekly maintenance doses. This regimen sensitizes the tumor cells to the anti-mitotic, anti-proliferative insult delivered by docetaxel. When docetaxel is administered with the eIF-4E ASO on day 21, the patient is first given a 1-3 hour intravenous infusion of the eIF-4E ASO, followed by administration of docetaxel within 30-60 minutes after the end of the eIF-4E ASO infusion. Docetaxel is conventionally administered via a 1 hour intravenous infusion. After the initial 21 days of treatment, this regimen can be repeated every 21 days over the course of therapy, employing further maintenance doses, but without further loading doses. Thus, subsequent therapeutically effective intervals after the initial 21 day therapeutically effective interval can be 21 days after administration of eIF-4E ASO/docetaxel combinations on the same day. As in the case of the amounts of the eIF-4E ASO and docetaxel effective in the combination therapeutic methods disclosed herein, a therapeutically effective interval for any individual patient undergoing treatment with the eIF-4E ASO and docetaxel can be determined by monitoring of a biomarker appropriate for the cancer. As noted above, a variety of different biomarkers useful in monitoring non-small cell lung cancer, prostate cancer, and breast cancer are available for this purpose. It should be noted that the 21 day intervals discussed above in connection with both the initial therapeutically effective interval and the subsequent therapeutically effective intervals represent typical starting procedures that are flexible and subject to modification. These intervals can be further optimized, and can be shorter or longer, the effectiveness of which can be monitored via the use of relevant biomarkers as noted above.

In another embodiment, treatment with the eIF-4E ASO and docetaxel can include a pre-docetaxel treatment period comprising administration of 3 loading doses of the eIF-4E ASO, i.e., a 1-3 hour infusion of the eIF-4E ASO once per day for 3 consecutive days during the first week, followed by administration of both the eIF-4E ASO and docetaxel as described above during the second week. Thus, the therapeutically effective interval in this case is about 4-7 days. This can be followed by administration of the eIF-4E ASO only during each of the next two succeeding weeks, followed by administration of the ASO and docetaxel again during the next week. Thus, docetaxel can be administered every approximately 14 days in conjunction with weekly administration of the eIF-4E ASO after the second week of therapy in which the eIF-4E ASO and docetaxel are both administered, for as long as treatment continues.

Thus, in view of the different treatment regimens discussed above comprising various intervals within which docetaxel can be administered after administration of the eIF-4E ASO, in particular embodiments, therapeutically effective intervals include about 4 days to about 21 days, about 4 days to about 7 days, about 14 days, and about 21 days after administration of the eIF-4E ASO alone. When the eIF-4E ASO and docetaxel are employed in the present in vivo methods, the effective interval can also be about 4 days to about 21 days, about 4 days to about 7 days, about 14 days, and about 21 days after administration of the eIF-4E ASO alone.

The eIF-4E ASO is used in the form of a pharmaceutically acceptable salt, preferably an alkali metal salt, more preferably a lithium, sodium, or potassium salt, most preferably a sodium salt. Such salts, and common methodology for preparing them, are well known in the art. See, e.g., P. Stahl et al. (2002) Handbook of Pharmaceutical Salts: Properties. Selecuon and Use, VCHA/Wiley-VCH: Berge et al. (1977) “Pharmaceutical Salts,” Journal of Pharmaceutical Sciences 66(1): 1-19. Docetaxel is a non-salt trihydrate.

The compounds of the present invention can be used as medicaments in human or veterinary medicine, administered by a variety of routes. Most preferably, such compositions are for parenteral administration, especially intravenous administration by slow infusion. Administration of solutions of these compounds, especially sterile injectable, non-pyrogenic solutions, by slow intravenous infusion is most preferred. Such pharmaceutical compositions can be prepared by methods well known in the art (See, e.g., Remington: The Science and Practice of Pharmacy. 19^{th} ed. (1995), A. Gennaro et al., Mack Publishing Co.), and comprise compounds of the invention and a pharmaceutically acceptable carrier, diluent, or excipient.

The following non-limiting example illustrates the present invention.

### EXAMPLE 1

### Reduction of Lung, Prostate, and Breast Tumor Volume in Mouse Xenografts by Combined Use of the eIF-4E ASO and Docetaxel

To determine whether the eIF-4E ASO complements or enhances the activity of docetaxel, the effect of the combined use of the eIF-4E ASO and docetaxel on tumor volume in 3 human xenograft cancer models is studied as follows.

Xenograft studies are carried out as described in Graff et al. (2005) Cancer Res. 65:7462-7469. Five million human cancer cells representing non-small cell lung cancer (line A549; ATCC, accession no. CCL-185), hormone refractory prostate cancer (line PC-3; ATCC, accession no. CRL-1435), and ER-/PR-HER2-breast cancer (line MDA-231; ATCC, accession no. HTB-26) are injected subcutaneously in the flank of 6-8 week old, athymic nude mice (Harlan, Indianapolis, Ind.) in a 1:1 mixture of serum-free growth medium and matrigel (Becton Dickinson, Bedford, Mass., catalogue #354234). Mice are monitored daily for palpable tumors.

When tumor volumes reach ˜50-100 mm³, mice are randomized to treatment groups (Graff et al. (2007) J. Clin. Invest. 117:2638-48). Body weight is monitored each time tumors are measured.

For treatment, all mice are injected intravenously with the eIF-4E ASO in saline in a total volume of 200 μl. The ASO consists of the nucleotide sequence 5′-TGTCATATTCCTGGATCCTT-3′ (SEQ ID NO:1), in which every internucleoside linkage is a phosphorothioate linkage, nucleotides 1-5 and 16-20 reading from the 5′ end to the 3′ end each comprise a 2′-O-(2-methoxyethyl) sugar, nucleotides 6-15 are 2′-deoxynucleotides, every cytosine residue is a 5-methylcytosine, and which is in the form of a sodium salt. The mice are first injected with an initial loading dose of 50 mg/kg ASO for 3 consecutive days, followed by 50 mg/kg thrice weekly thereafter.

Taxotere® (docetaxel) dosing begins 8 days after the first ASO dose to allow time for reduction of eIF-4E protein expression. Mice are dosed with 2.5 or 10 mg/kg Taxotere® (Sanofi-Aventis) intraperitoneally (IP) in 1% ethanol in 5% dextrose water in a volume of 200 μl once weekly for 3 weeks.

All animal work is performed with Institutional Animal Care and Use Committee approval in an AALAC-certified facility.

Tumor volumes are calculated measuring the largest (L) and smallest (W) diameters with a caliper. The formula is: Volume=L×W²×0.534. Tumor volume data are transformed to a log scale to equalize variance across time and treatment groups. The log volume data are analyzed with a two-way repeated measures analysis of variance by time and treatment using SAS PROC MIXED software (SAS Institute Inc, Cary, N.C.) using the spatial power covariance structure. Treatment groups are compared to the control group at each time point and overall. The data may be plotted as means and standard errors for each treatment group versus time. Additivity is assessed in two ways: (1) as an overall interaction effect between the ASO and Taxotere® in the repeated measures analysis, and (2) by using the Bliss Independence method on the final day of the study (C.I. Bliss (1939) Ann. Appl. Biol. 26:585-615). These two methods yield the same results.

The results are presented in Tables 1-3, below. The observed percent reductions in mean tumor volume at the conclusion of each study resulting from the various treatments are shown in Table 4. These are calculated as:

$$\frac{\begin{matrix}Vehicle Control Mean Tumor Volume - \\ Treatment Mean Tumor Volume\end{matrix}}{Vehicle Control Mean Tumor Volume} \times 100$$

The “Expected if Additive” percent reductions in mean tumor volume shown in Table 4 are calculated using the Bliss Independence definition of additivity, as follows:

$$\frac{\begin{matrix}ASO Mean Tumor Volume \times \\ Docetaxel Mean Tumor Volume\end{matrix}}{Vehicle Control Mean Tumor Volume}$$

TABLE 1
A549 Non-Small Cell Lung Cancer Xenograft Tumor Volumes

| | | | | | | | eIF-4E ASO + | |
|---|---|---|---|---|---|---|---|---|
| | Vehicle | | | | Docetaxel | | Docetaxel | |
| | Control | | eIF-4E ASO | | (10 mg/kg) | | (10 mg/kg) | |
| | Mean | | Mean | | Mean | | Mean | |
| A549 | Tumor | Std. | Tumor | Std. | Tumor | Std. | Tumor | Std. |
| Day | Volume | Error | Volume | Error | Volume | Error | Volume | Error |
| 7 | 54.9 | 12.2 | 52.7 | 8.7 | 49.7 | 7.9 | 52.8 | 8.2 |
| 10 | 65.6 | 14.5 | 69.4 | 11.5 | 56.7 | 9.0 | 59.7 | 9.2 |
| 15 | 115.1 | 25.5 | 113.7 | 18.8 | 114.8 | 18.2 | 105.5 | 16.3 |
| 18 | 75.8 | 16.8 | 79.1 | 13.1 | 70.0 | 11.1 | 68.5 | 10.6 |
| 22 | 143.0 | 31.6 | 139.5 | 23.0 | 135.8 | 21.6 | 85.5 | 13.2 |
| 25 | 176.7 | 39.1 | 149.0 | 24.6 | 170.1 | 27.0 | 105.4 | 16.3 |
| 29 | 242.8 | 53.7 | 220.1 | 36.3 | 257.0 | 40.8 | 136.9 | 21.2 |
| 32 | 276.8 | 61.3 | 249.5 | 42.2 | 261.5 | 41.5 | 130.1 | 20.1 |
| 36 | 362.0 | 80.1 | 256.6 | 43.5 | 290.0 | 46.0 | 155.9 | 24.1 |
| 39 | 351.2 | 77.7 | 239.1 | 40.8 | 282.3 | 44.8 | 157.9 | 24.4 |
| 43 | 422.7 | 93.6 | 313.2 | 53.8 | 342.6 | 54.4 | 155.7 | 24.5 |
| 46 | 446.4 | 98.8 | 292.3 | 50.4 | 310.2 | 51.0 | 171.2 | 28.1 |
| 50 | 488.2 | 108.0 | 404.0 | 69.8 | 343.8 | 58.2 | 214.5 | 36.5 |

TABLE 2
PC-3 Hormone-Refractory Prostate
Cancer Xenograft Tumor Volumes

| | | | | | | | eIF-4E ASO + | |
|---|---|---|---|---|---|---|---|---|
| | Vehicle | | | | Taxotere ® | | Taxotere ® | |
| | Control | | eIF-4E ASO | | 10 mg/kg | | (10 mg/kg) | |
| | Mean | | Mean | | Mean | | Mean | |
| PC3 | Tumor | Std. | Tumor | Std. | Tumor | Std. | Tumor | Std. |
| Day | Volume | Error | Volume | Error | Volume | Error | Volume | Error |
| 7 | 49.7 | 9.6 | 49.8 | 9.3 | 46.3 | 8.7 | 50.1 | 10.6 |
| 12 | 97.5 | 18.8 | 88.3 | 16.5 | 106.8 | 20.0 | 75.5 | 15.9 |
| 15 | 125.2 | 24.1 | 130.3 | 24.3 | 134.7 | 25.3 | 103.5 | 21.8 |
| 19 | 173.2 | 33.4 | 159.0 | 29.7 | 164.8 | 30.9 | 126.9 | 26.8 |
| 22 | 221.4 | 42.6 | 218.8 | 40.8 | 171.7 | 32.2 | 148.5 | 31.4 |
| 27 | 296.4 | 57.1 | 294.5 | 54.9 | 157.4 | 29.5 | 117.9 | 24.9 |
| 29 | 368.7 | 71.0 | 343.9 | 64.2 | 162.7 | 30.5 | 107.7 | 22.7 |
| 33 | 463.2 | 89.2 | 424.0 | 79.1 | 161.6 | 30.3 | 106.0 | 22.4 |
| 35 | 546.8 | 105.3 | 491.2 | 91.6 | 167.0 | 31.3 | 91.5 | 19.3 |

TABLE 3
MDA-231 Breast Cancer Xenograft Tumor Volumes

| | | | | | | | eIF-4E ASO + | |
|---|---|---|---|---|---|---|---|---|
| | Vehicle | | | | Taxotere ® | | Taxotere ® | |
| | Control | | eIF-4E ASO | | (2.5 mg/kg) | | (2.5 mg/kg) | |
| MDA- | Mean | | Mean | | Mean | | Mean | |
| 231 | Tumor | Std. | Tumor | Std. | Tumor | Std. | Tumor | Std. |
| Day | Volume | Error | Volume | Error | Volume | Error | Volume | Error |
| 5 | 77.7 | 7.2 | 78.9 | 7.3 | 78.7 | 5.6 | 79.6 | 10.6 |
| 8 | 124.1 | 11.5 | 129.4 | 11.9 | 130.1 | 9.3 | 127.0 | 16.9 |
| 12 | 194.4 | 18.0 | 166.6 | 15.3 | 180.9 | 12.9 | 203.3 | 27.1 |
| 15 | 248.3 | 22.9 | 221.0 | 20.3 | 221.0 | 15.8 | 205.3 | 27.3 |
| 19 | 332.5 | 30.7 | 268.2 | 24.7 | 270.3 | 19.3 | 208.5 | 27.8 |
| 22 | 389.4 | 36.0 | 356.3 | 32.8 | 333.9 | 23.9 | 234.3 | 31.2 |
| 26 | 438.9 | 40.5 | 435.9 | 40.1 | 384.9 | 27.5 | 249.5 | 33.2 |
| 29 | 507.0 | 46.8 | 574.1 | 52.8 | 450.4 | 32.2 | 263.0 | 35.0 |
| 33 | 536.9 | 49.6 | 626.2 | 57.6 | 529.6 | 37.9 | 292.8 | 39.0 |

TABLE 4
Percent Reduction in Mean Tumor
Volumes Due to Various Treatments

| | | | | ASO + | |
|---|---|---|---|---|---|
| | | | | Taxo- | ASO + |
| | | | | tere ® | Taxo- |
| Tumor Type | Vehicle | | Taxo- | Expected if | tere ® |
| And Day | Control | ASO | tere ® | Additive | Observed |
| NSCLC | 0% | −17.3% | −29.6% | −41.7% | −56.1%* |
| A 549 | | | | | |
| Day 50 | | | | | |
| Prostate | 0% | −10.2% | −69.5%* | −72.6% | −83.3%* |
| PC3 | | | | | |
| Day 35 | | | | | |
| Breast | 0% | +16.6% | −1.4% | +15.0% | −45.5%*^{#} |
| MDA-231 | | | | | |
| Day 33 | | | | | |

*p < 0.05 vs. Vehicle Control
^{#}p < 0.05 for combination effect greater than additive.

The data in Tables 1-3 reveal that groups treated with the combination of the eIF-4E ASO and docetaxel exhibit the most substantial, statistically significant (*p<0.05 vs. vehicle control) reduction in tumor growth. The combination of the eIF-4E ASO and docetaxel produces substantial reduction in tumor growth in the 3 distinct human tumor xenografts representing non-small cell lung cancer (A549), hormone refractory prostate cancer (PC-3), and ER-/PR-/HER2-breast cancer (MDA-231) compared to the reduction resulting from each drug alone.

In terms of the percent reduction in tumor volume by the end of each study, as shown in Table 4, treatment with the eIF-4E ASO in combination with docetaxel results in an additive effect in the prostate cancer and non-small cell lung cancer tumor models. In each of these cases, the treatment effect resulting from the combination is better than additive, i.e., −56.1% is better than −41.7% and −83.3% is better than −72.6%, but not statistically significantly better than additive.

In the breast cancer model, treatment with the ASO alone results in a non-statistically significant increase in mean tumor volume, while treatment with the combination of the ASO and docetaxel produces a significantly greater-than-additive reduction in mean tumor volume. The asterisk indicates that −45.5% is statistically different from 0% (the vehicle control), whereas the pound sign indicates that −45.5% is statistically different from +15% (expected if additive), showing that there is statistically significant synergy in the breast cancer cell line.

In all cases, treatment with the eIF-4E ASO sensitizes the tumor cells to the anti-mitotic, growth-, and proliferation-inhibiting action of docetaxel.

1. A method of treating prostate cancer comprising administering to a patient in need thereof a therapeutically effective combination of docetaxel and a modified eIF-4E antisense oligonucleotide which is in the form of a pharmaceutically acceptable salt, wherein said docetaxel is administered after administration of said modified eIF-4E antisense oligonucleotide within a therapeutically effective interval, thereby treating prostate cancer in the patient.

2. The method of claim 1, wherein said therapeutically effective interval is in the range of from about 4 days to about 21 days.

3. The method of claim 2, wherein the modified eIF-4E antisense oligonucleotide is a compound of formula I: 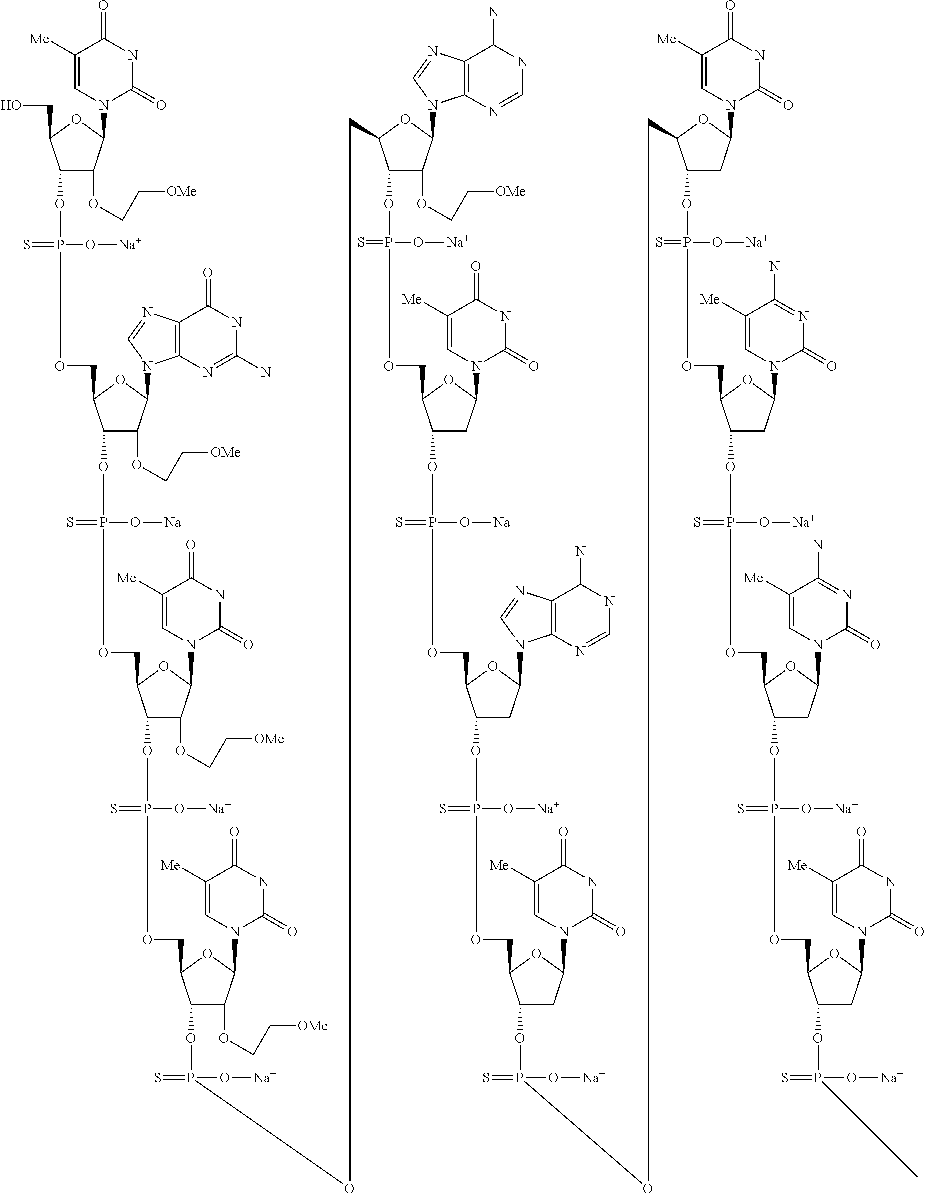 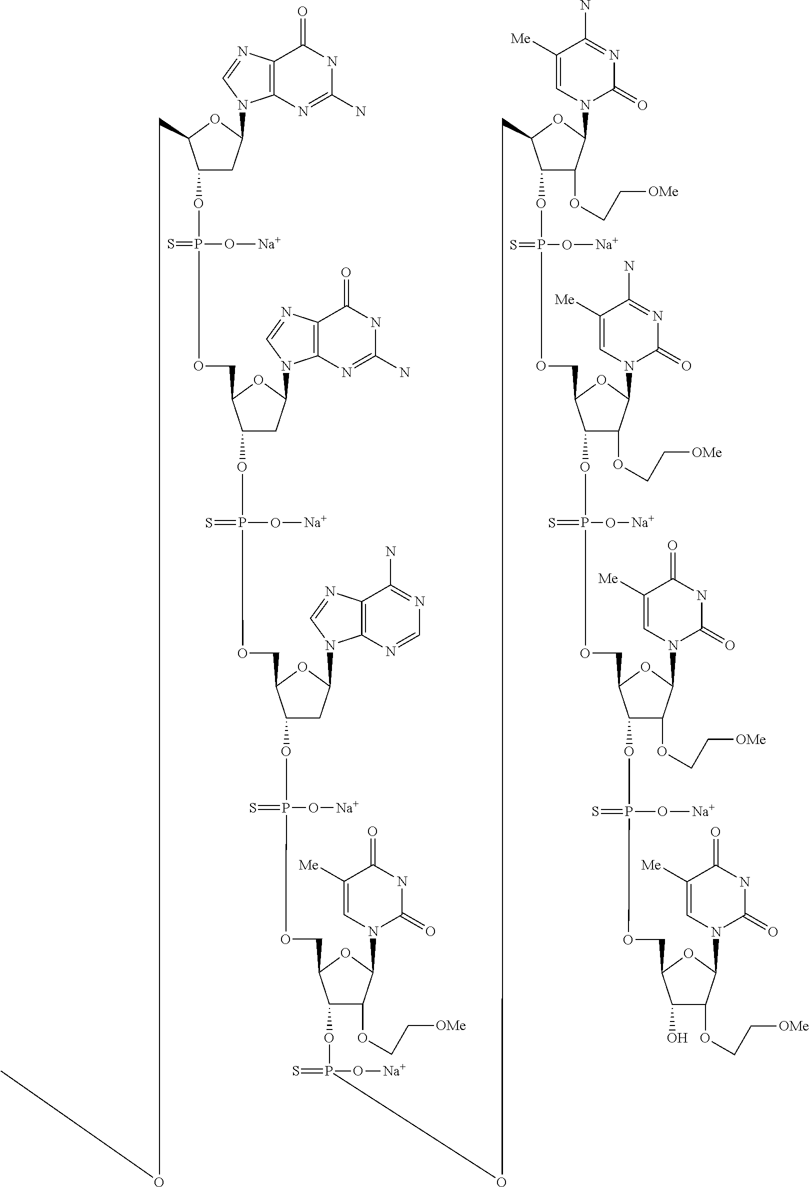
or other pharmaceutically acceptable salt thereof.

4. The method of claim 3, wherein said combination therapy is via the parenteral route.

5. The method of claim 3, wherein said combination therapy is via intravenous administration.

6. The method of claim 5, wherein said intravenous administration is via slow infusion.
